(19) United States
(12) Patent Application Publication
(10) Pub. No.: US 2006/0224185 A1
(43) Pub. Date: Oct. 5, 2006

(54) Endogeneous alpha-MSH treatment for stroke

(76) Inventors: Thomas M. DiMauro, Southboro, MA (US); Mohamed Attawia, Canton, MA (US); Sean Lilienfeld, Sharon, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/096,085

(22) Filed: Mar. 31, 2005

(51) Int. Cl. A61N 1/39

(52) U.S. Cl. 607/1

(57) ABSTRACT

Heating the hypothalamus, preferably by ultrasound, to cause the release of αMSH in the brain.

### BACKGROUND OF THE INVENTION

There are about 500,000-700,000 strokes per year in the United States. In addition to the loss of quality of life, it has been estimated that strokes cost the economy billions of dollars per year.

It is now known that stroke pathology is not a singular event, but rather a succession of events that causing progressive damage. At the onset of a stroke, one portion of the brain becomes infracted. The infarction results in a release of neurotoxic amounts of glutamate from the dying cells, thereby causing adjacent brain surrounding the infracted portion (the “penumbra”) to become at risk. However, the brain cells in the penumbra may still be saved. Within hours of the stroke, inflammatory cells invade the penumbra, exposing the penumbra to more neurotoxins such as TNF-α and Il-1β. Accordingly, modern stroke therapy has focused upon saving the brain cells in the penumbral region from these neurotoxic agents

It has been reported by Deng, Stroke, 2003, 34:2495-2501 that mild hypothermia reduces cerebral injury in the laboratory, and that this protection has been attributed to preservation of metabolic stores and decreases in excitatory amino acid (glutamate) release. It is further thought that mild hypothermia may protect by other mechanisms as well because it protects even when delayed for hours after ischemic onset when excitatory amino acids have been released and energy stores have been exhausted. Some studies indicate that the acute inflammatory response contributes significantly to injury after ischemia, and that protection by mild hypothermia is associated with anti-inflammatory processes. Deng further reported that mild hypothermia decreases inflammatory responses in both stroke and brain inflammation, implicating a direct anti-inflammatory effect of cooling, and suggesting that hypothermia can attenuate factors contributing to delayed ischemic injury.

There are presently a number of conventional cooling therapies that have been reported by the literature. Cooling blankets have been used to attenuate stroke and improve recovery by 15%-36% (Clifton-Houston). Kotulak, “Inside the Brain”, 1997, Andrews McMeel Publishing: Kansas City, p. 175. Commercially available microcatheters have been used to locally induce endovascular hypothermia. Slotboom, Neuroradiology, 2004, Nov. 46(11) 923-34. Cooling helmets have also been studied. However, each of these approaches suffers from either being an invasive approach (which is generally impractical during a stroke situation) or relying upon external cooling (which is slow).

Therefore, it is one object of the present invention to develop a therapy for treating stroke that is both non-invasive and quick.

### SUMMARY OF THE INVENTION

Alpha melanocyte stimulating hormone (αMSH) is a hormone produced mainly in the pituitary gland and functions as a control of skin pigmentation. This molecule, produced by post-translational processing of pro-opiomelanocortin (POMC), is a 13 amino acid peptide highly conserved across phylogeny and widely expressed in tissues. Eberle A N, The Melanotropins, Basel (ed. S. Karger) 1988. The peptide is produced by the pituitary and by many extrapituitary cells, including monocytes, astrocytes, gastrointestinal cells, and keratinocytes. Endogenous αMSH modulates fever and inflammation.

The present inventors have noted a number of additional qualities about αMSH pertinent to the present invention:

First, endogenous αMSH is produced not only in the pituitary gland for release into the blood stream, it is also produced by the hypothalamus. Huang, J. Neurosci., 17(9), May 1, 1997, 3343-51 reports that αMSH-containing fibers are found emanating from the arcuate nucleus portion of the hypothalamus—its sole source in the forebrain. Tatro, Clin. Infect. Dis. 2000, 31: S190-201, reports that endogenous αMSH is synthesized within the brain and acts at target sites in the CNS as critical modulators of diverse autonomic functions including thermoregulation. Tatro, supra, further teaches that the principal αMSH-producing neuron group of the CNS is located in the arcuate nucleus of the medial basal hypothalamus, from which it projects to innervate numerous forebrain and brain stem centers involved in neuroendocrine and autonomic function.

A number of investigators have reported that αMSH can be released from the hypothalamus via its depolarization. See, e.g., O'Donohue, Peptides, 1981, Spring 2(1) 93-100; Jegou, Brain. Res., 1987, Jun 16, 413(2) 259-66; Bunel, Brain. Res., 1990, Apr. 16, 513(2) 299-307; Tranchand, Brain Res. Mol. Brain Res., 1989 Jul. 6, 6(1) 21-9; In particular Bunel, Brain. Res., 1990, Apr. 16, 513(2) 299-307 reports that αMSH can be released into the hippocampus by depolarization of the hypothalamus.

Second, αMSH also has anti-pyretic qualities and is released during fever. Tatro, Clin. Infect. Dis. 2000, 31: S190-201. The literature has further reported that αMSH can be used to quell an IL-1β-induced fever. This means that inducing a fever in a patient will have the effect of releasing MSH throughout the brain.

Third, the CNS possesses temperature sensors located throughout brain tissue, but located mainly in the septal region and the pre-optic region of the hypothalamus. Tatro, supra CID, teaches that projections of αMSH-producing neurons are particularly dense in the septal and ventromedial preoptic regions, which are critical thermoregulatory centers. Samson, Peptides, 1981 Winter 2(4), 419-23, and Bell, Am. J. Phys., 1987 June 252 (6Pt.2) R1152-7 each report that αMSH is primarily released into the septum in a pulsatile fashion during fever. Samson, Peptides, 1981 Winter 2(4), 419-23, and Holdeman, Am. J. Physiol. 1985, Jan. 248(1Pt.2) R125-9 each report that significiantly higher levels of αMSH are found in the septal regions of febrile animals than in control animals. Glyn-Ballinger, Peptides, 1983, Mar-Apr. 4 (2) 199-203 concludes that septal neurons are important to the central modulation of body temperature. Feng, Brain Res. Bull., 1987, Ap 18(4) 473-7 reports that the pre-optic region of the brain is the primary temperature control of the CNS.

This means that a temperature increase needs to be induced in only a small portion of the brain (i.e., either the septum or the pre-optic region of the hypothalamus) in order to evoke an αMSH release to the septum.

Therefore, given the pivotal role played by αMSH in abating fever and the ability of the arcuate nucleus to distribute αMSH to the septum in response to a temperature increase, the present inventors have developed methods of inducing endogenous αMSH release throughout the brain. The present inventors have derived therapies based upon selectively heating a portion of the brain in order to induce the release of a therapeutic amount of αMSH by the brain.

Therefore, in accordance with the present invention, there is provided a method of providing therapy for a neurodegenerative disease, comprising the steps of:
a) selectively heating a portion of a brain of a patient having a CNS disease (such as a stroke patient) to induce intracerebral cooling.

In some embodiments, there is provided the use of non-invasive methods (such as ultrasound) to heat selected thermosensitive neurons in the brain (such as are in the hypothalamus) so that the brain responds to fever-like conditions and releases αMSH to the septum. New ultrasound methods can heat small portions of the brain (such as the hypothalamus) a few degrees. Upon heating, the arcuate nucleus portion of the hypothalamus will release αMSH to the septum and pre-optic regions, each of which are thermoregulatory centers for the brain. It is believed that these thermoregulatory centers will become active, inducing a beneficial response in the patient's brain. The reduced temperature will be neuroprotective, due to inhibition of glutamate release and reduction of inflammation. This therapy could be useful for stroke, and for any neurodegenerative disease having an inflammatory component, such as MS or AD.

The pre-selected heating theory of the present invention is supported by many studies investigating thermoregulation that used thermodes to control the temperature of discrete areas of the hypothalamus. In Magoun, J. Neurophysiol. 1938, 1:101-114, localized hypothalamic heating was used to evoke panting in anesthetized cats. In Hemingway, J. Neurophysiol., 1940, 3:329-38, localized hypothalamic warming suppressed ongoing shivering and evoked ear vasoldilation. Preoptic warming has been shown to elicit cutaneous vasodilation, sweating, panting and various behavioural responses that enhance heat loss. Boulant, “Handbook of the Hypothalamus”, 3(A), New york: Marcel Dekker, 1-82; Freeman, Am. J. Physiol. 1959, 197, 145-8; Boulant, Brain Res. 1977, 120, 367-72; Kanosue, Am. J. Physiol. 1994, 267:R283-8; Kanusue, Am. J. Physiol. R 275-82; Gisolfi, Brain Res. Bull., 1988, 20, 179-82. Thus, it is reasonable to expect that selective heating of the hypothalamus will engender a heat loss response in the brain as well.

The present invention thus has advantages over conventional methods of treating stroke in that the therapy is non-invasive, local and quick-acting.

### DETAILED DESCRIPTION OF THE INVENTION

In general, the amount of heat selectively applied to a portion of the brain should be substantial enough to induce a fever-like response, but not enough to cause damage to the bran tissue. Accordingly, in preferred embodiments, the heating is effective to produce a temperature rise of between about 1° C. and 2° C. in the heating target portion of the brain. In general, the heating of the targeted portion of the brain should not produce a temperature rise in the affected region above about 42° C. It is preferred that the targeted portion of the brain be heated for a period of between about 1 minute and about 24 hours.

In some embodiments, the desired temperature rise is effectuated in the septal region of the brain. In others, the desired temperature rise is effectuated in the pre-optic region of the brain. In others, the desired temperature rise is effectuated in the hippocampal region of the brain. In others, the desired temperature rise is effectuated in the amygdala region of the brain. In some embodiments, more than one of these regions is heated.

In general, the amount of cooling experienced by the brain due to the antipyretic release should be substantial enough to attenuate the attack of the penumbral region of the infracted tissue, but not enough to cause damage to the bran tissue. Accordingly, in preferred embodiments, the cooling is effective to produce a temperature drop of between about 0.1° C. and 10° C. in the penumbral portion of the brain, more preferably between 0.5° C. and 5° C.

As the heating of the hypothalamus is expected to quickly cause the release of αMSH, significant release of αMSH is expected to occur within about one hour, and preferably less than about 30 minutes after heating is begun, more preferably less than 10 minutes. As αMSH release from the hypothalamus is expected to cause a relatively quick response, the recited temperature drops are expected to occur within about one hour, and preferably less than about 30 minutes after heating is begun, more preferably less than 10 minutes.

In preferred embodiments, the selected heating is carried out by non-invasive, transcerebral ultrasound heating. Since this therapy is non-invasive, the risk of potential complications and patient recovery issues associated with implanting heating devices in the brain are avoided.

Historically, transcranial ultrasound heating therapies have been avoided due to the high distortion and energy absorption associated with the bone of the skull. Recently however, there have been numerous reports in the literature that these problems have been solved. In particular, new transcerebral ultrasound technology is able to precisely and accurately heat selective portions of the brain. In one particular report, Hynynen, Magnetic Resonance in Medicine, 52:100-107(2004), the investigators were able to produce a 39° C. degree peak in an experimental set up comprising an exposed rat brain located within a water-filled human skull. According to the authoers, recent advances in transducer, amplifier and medical imaging technology as well as progress in ultrasound modeling have increased the feasibility of using focused ultrasound for noninvasive brain therapy. Therefore, it is reasonable to expect that straightforward modification of this apparatus can provide for its use in patients.

In some embodiments, the transcranial ultrasound technology disclosed in U.S. Pat. No. 6,770,031, entitled “Ultrasound Therapy” (Hynynen), the specification of which is incorporated by reference in its entirety, is used. Other articles disclosing suitable transcranial ultrasound technology for use in the present invention include Sun, J. Acoust. Soc. Am., 1999, Apr 105(4) 2519-27; Sun, J. Acoust. Soc. Am., 1998, Sept. 104(3 Pt.1):1705-15; Connor, IEEE Trans. Biomed. Eng. 2004 October 51(10) 1693-706; Clement, Phys. Med. Biol. 2002, Apr 21; 47(8), 1219-36; Hynenen, Ultrasound in Med. & Biol. 24(2) 275-283 (1998); Nynynen, Neuroimage., 2005 Jan. 1, 24(1) 12-20; Clement, Phys. Med. Biol., 2000 December 45(12) 3707-19; Clement, Phys. Med. Biol. 2000 April 45(4) 1071-83; the specifications of which are incorporated by reference in their entirety.

In some embodiments, a biocompatible antenna is implanted in a portion of the brain (preferably near the septum or preoptic region) and energized by microwaves in order to produce the desired heating. Articles disclosing suitable transcranial microwave technology for use in the present invention include Wu, Med. Phys. 1987, Mar-Apr. 14(2) 235-7; Satoh, Neurosurgery, 1988, Nov 23(5) 564-7. Ryan, Int. J. Radiat Oncol. Biol. Phys. 1991, Apr. 20(4), 739-50.

In some embodiments, electrodes are implanted in a portion of the brain (preferably near the septum or preoptic region) and energized in order to depolarize the hypothalamus, and provoke MSH release.

In some embodiments, a biocompatible resistance wire is implanted in a portion of the brain (preferably near the septum or preoptic region) and a voltage is applied across its electrodes in order to produce the desired heating. Articles disclosing suitable intracranial electrode technology for use in the present invention include Baker, J. Magn. Reson. Imaging 2004 Aug. 20(2) 315-20.

In some embodiments, a proton beam is directed towards a portion of the brain (preferably near the septum or preoptic region) in order to produce the desired heating. Articles disclosing suitable transcranial proton beam technology for use in the present invention include Brisman, Neurosurgery, 53:951-962 (2003).

Because neurons are extremely sensitive to temperature, care should be taken not to exceed preselected temperature levels. Therefore, in some embodiments, a temperature monitoring system is employed in order to assure that the appropriate temperatures are being produced (i.e., a warm, but not too hot hypothalamus and a cool cerebral cortex). Articles disclosing suitable transcranial temperature monitoring for use in the present invention include Sherar, Phys. Med. Biol. 2000 December 45(12) 3563-76.

Without wishing to be tied to a theory, it is believed that selective heating of thermosensitive neurons in the brain to effect αMSH release may also have therapeutic value for the treatment of other diseases. One possible candidate is Alzheimer's Disease.

In Alzheimer's Disease (AD), the cleavage of beta amyloid protein precursor from the intracellular membrane often produces a protein AB-42 which is incompletely removed by normal clearance processes. Over time, this protein is deposited as a beta amyloid protein Aβ plaque within brain tissue, leading to the local destruction of neurons. The Aβ plaque deposition is also believed to provoke an inflammatory response by microglia and macrophages, which recognize the plaque as a foreign body. These cells are believed to respond to the plaque deposition by releasing pro-inflammatory cytokines and reactive oxygen species (ROS). Although the inflammatory response may be provoked in an effort to clear the brain tissue of the detrimental plaque, it is now believed that this inflammation also injures local neuronal tissue, thereby exacerbating AD.

One key set of neurons damaged by AD are the septohippocampal neurons, which are cholinergic neurons. Arai, Brain Research, 377 (1986), 305-310, characterizes damage of these neurons as “one of the important pathogenetic factors” of AD.

Without wishing to be tied to a theory, it is believed that selective release of MSH into the septum will have a beneficial therapeutic effect upon cholinergic septohippocampal neurons. Each of Botticelli, Nature, 1981, Jan. 1, 289 (5793) 75-76 and Wood, J. Pharmacol. Exp. Ther. 209(1978) 97-103 have investigated whether MSH from the septum can influence acetylcholine turnover from the septohippocampal neurons. Since MSH has a beneficial effect upon acetylcholine turnover, it is believed that the influence of MSH from the septum upon septohippocampal neurons that are under attack will have a beneficial neuroprotective effect upon these vital neurons.

In addition to the beneficial effect provided by MSH to the septohippocampal neurons during AD, it is possible that the selective heating of the hypothalamus may cause the release of MSH to other portions of the brain, namely that hippocampus and the amygdala, to provide a neuroprotective effect as well. The present inventors are not aware of any study that investigated the possible release of MSH to the hippocampus and the amygdala during fever. However, there are two good reasons for believing that MSH may be released to these areas in an attempt to quell a fever.

First, it has been reported each of these areas contain MSH-containing fibers. Jegou, Brain Res. Mol. Brain Res. 1989 Jul. 6, 6(1) 21-9 reports that αMSH is synthesized by discrete populations of hypothalamic neurons which project into different regions of the brain, including the cerebral cortex, the hippocampus and the amygdala nuclei. O'Donohue, Prog. Biochem. Pharmacol., 1980, 16, 69-83, reports that alpha-MSH-containing fibers were present in various nuclei of the hypothalamus, preoptic area, septum, amygdala, mammillary body and central gray area. Kohler, J. Comp. Neurol., 1984, Mar. 10, 223(4):501-14 teaches the existence of diffuse αMSH projections to the hippocampus from individual neurons of the hypothalamus, and reports some such fibers reach the hippocampus through the septal route. Bunel, Brain. Res., 1990, Apr. 16, 513(2) 299-307 reports that αMSH can be released into the hippocampus by depolarization of the hypothalamus.

Second, each of these areas also contain thermosensitive neurons. Bachtell, Brain Research, 960(2003) 157-164.

Since each of these areas is involved in the thermoregulatory action in the brain and each has the capacity the accept MSH released from the hypothalamus, it is quite possible that the hypothalamus releases MSH into the hippocampus and amygdala in response to a local heating. Accordingly, the selective heating of the hypothalamus may provide physiologically significant levels MSH to the hippocampus and amygdala.

The ability of the brain to provide MSH to the hippocampus and amygdala is important because MSH is well known for its anti-inflammatory properties.

aMSH has been extensively linked to immunosuppression and tolerance. Lipton, News Phys. Sci., 15, 2000, 192-5 reports that αMSH is an important neuroimmunomodulator. Luger, NY Acad. Sci. 1999, Oct. 20, 885, 209-16 reports that αMSH plays an important role in light-mediated immunosuprression.

The literature has extensively reported that αMSH upregulates anti-inflammatory processes. For example, Luger, Ann. NY Acad. Sci., 2003 Jun. 994:133-40 reports that a αMSH modulates antigen presenting function and upregulates IL-10, a known anti-inflammatory cytokine. Taylor, Immunol. Cell Biol., 2001 Aug. 79(4) 358-67 reports that αMSH induces the production a TGF-B, a known anti-inflammatory cytokine, by T cells.

The literature has also extensively reported that αMSH antagonizes the activity of pro-inflammatory molecules, and it is believed that αMSH is involved in the regulation of the NFkB pathway of pro-inflammatory cytokine production. Luger, Pathobiology, 1999, 67(5-6), 318-21 reports that αMSH functions to downregulate NFκB activity, thereby downregulating pro-inflammatory cytokine production. Luger concludes that αMSH plays an essential role in inducing tolerance. The inhibition of IL-1B-induced acute inflammation by MSH analogs has also been reported in the literature. See Hiltz, Cytokine, 1992, Jul 4 (4): 320-8; Ceriani, Neuroendocrinology, 1994, Feb. 59(2):138-43; and Watanabe, Brain. Bull. Res., 1993, 32(3):311-4. Wong, Neuroimmunomodulation, 1997 January-February 4(1), 37-41 reports the modulation of TNF-a production within an inflamed brain by MSH, and concludes that aMSH represents a potential local mechanism of anti-inflammatory action within the brain. MSH inhibits the inflammatory cascade at many sites: it reduces production of NO (Star, Proc Natl Acad Sci USA 1995; 92:8016-8020), proinflammatory cytokines (Lipton, Immunol Today 1997; 18:140-145), monocyte chemoattractant protein 1 (MCP-1), and interleukin 8 (IL-8), and markedly decreases neutrophil chemotaxis in vivo and in vitro (Mason, J Immunol 1989; 142:1646-1651; Catania, Peptides 1996; 17:675-679.

These effects of the peptide are exerted, at least in part, through inhibition of activation of the nuclear factor NF-.kappa.B, a pivotal transcription factor for genes that encode proinflammatory cytokines, chemokines, and adhesion molecules. Manna, J Immunol 1998; 161:2873-2880; Ichiyama, Exp Neurol 1999; 157:359-365.

See also a review in Lipton, “Marshalling the Anti-Inflammatory Influence of the Neuroimmunomodulator MSH”, News Physiol. Sci., Vol. 15, August 2000, pp. 192-195. Lipton states that MSH inhibits TNF-a and may limit neurodegeneration and other CNS disorders that have an inflammatory component.

The literature has further appreciated the potential therapeutic possibilities of cerebrally administering αMSH in order to quell damaging brain-related inflammatory processes. Ichiyama, Brain. Res., 1999, Jul 31, 836 (102) 31-7, reports that systemic administration of αMSH inhibits brain inflammation, and does so by inhibiting the NFKB pathway. Rajora, J. Neurosci., 19997, Mar 15, 17(6) 2181-6 reports that αMSH inhibits brain inflammation by modulating TNF-a. Galimberti, Biochem. Biophys. Res. Comm., 1999 Sep. 16, 263(1) 251-6 examined a possible role for MSH in mediating Alzheimer's Disease and reported that αMSH inhibits NO and TNFa by microglia activated with BAP.

The literature has reported lower than normals levels of MSH in selected portions of the AD brain. Rainero, Neurology, 1988, Aug. 38(8) 1281-4. Arai, Brain Res. 1986, Jul 9, 377(2), 305-10, reports that lowers levels of MSH in AD patients in the following areas of the brain: the cingulated cortex, the caudate and the substantia nigra. Anderson, Med. Hypotheses, 1986, Apr. 19(4) 379-85 hypothesizes that αMSH deficiency may be a cause of Alzheimer's Disease.

Because of its potent immunosuppressive and anti-inflammatory qualities, αMSH has been linked to Alzheimer's Disease. Galimberti, supra, concludes that αMSH could modulate inflammation in senile plaques of AD brain, and that αMSH might be useful int eh treatment of AD given its inhibitory properties on AB-mediated inflammatory processes. XENGEN has proposed that αMSH be used therapeutically to treat Alzheimer's Disease.

It is believed that the release of MSH into the hippocampus and amygdala may have special benefit for the AD patient because the hippocampus and amygdala are often early locations in the brain that are damaged by AD. AD is a progressive disease that begins in the hippocampus, extends to the amygdala, and then proceeds anteriorly to the prefrontal cortex and posteriorly as well. By providing anti-inflammatory MSH to each of the hippocampus and amygdala at an early stage in the disease, the present invention may be able to attenuate the inflammation associated with AD so that the disease does not spread to the remainder of the brain.

In general, the amount of cooling experienced by the hippocampus and/or amygdala due to the antipyretic release should be substantial enough to attenuate the inflammatory attack of these regions, but not enough to cause damage to these tissues. Accordingly, in preferred embodiments, the cooling is effective to produce a temperature drop of between about 0.1° C. and 10° C. in the hippocampus or amygdala, more preferably between 0.5° C. and 5° C.

In summary, the present invention provides a non-invasive method of selectively heating thermosensitive neurons within the brain in order to mimic and fever that exceeds the set point of the hypothalamus and evoke a cooling response throughout the brain. The resultant cooling of the brain will likely have tremendously beneficial effects for patients suffering from stroke or from neurodegenerative diseases such as Alzheimer's Disease.

In some embodiments, the heating causes the release of MSH into the septum and thereby influences cholinergic turnover in the septohippocampal neurons. The resultant change in turnover will likely have tremendously beneficial effects for patients suffering from Alzheimer's Disease.

In other embodiments, the heating causes the release of anti-inflammatory molecules such as MSH into portions of the brain (such as the hippocampus or amygdala) that are under attack from inflammatory processes. The resultant attenuation of inflammation in the local tissue will likely have tremendously beneficial effects for patients suffering from neurodegenerative diseases such as Alzheimer's Disease.

Although the above devices offer the advantage of requiring only non-invasive procedures, there may be situations wherein multiple invasions of the skull are considered acceptable. In those situations, it may be acceptable to simply deliver exogenous αMSH locally to the inflamed tissue. If multiple invasions are considered acceptable, then, in another aspect of the present invention, there is also provided a method of treating or preventing a CNS disease, comprising the step of:

a) Intracerebroventricularly Administering an Effective Amount of αMSH.

Intracerebroventricularly administration of an effective amount of αMSH is a desirable procedure because the surgeon may conveniently obtain and deliver exogenous αMSH and thereby not rely upon the patient's hormonal system to produce the αMSH. Furthermore, intracerebroventricularly administration of an effective amount of αMSH can be conveniently carried out via an injection through the skull of the patient.

In some embodiments, the intracerebroventricularly administered αMSH is provided in a sustained release device. The sustained release device is adapted to remain within the brain tissue for a prolonged period and slowly release the αMSH contained therein to the surrounding environment. This mode of delivery allows an αMSH to remain in therapeutically effective amounts within the brain tissue for a prolonged period.

In some embodiments, the αMSH is predominantly released from the sustained delivery device by its diffusion through the sustained delivery device (preferably, though a polymer). In others, the αMSH is predominantly released from the sustained delivery device by the biodegradation of the sustained delivery device (preferably, biodegradation of a polymer). In some embodiments thereof, the implant comprises αMSH embedded in a polymer, such as PLA.

Preferably, the sustained release device comprises a bioresorbable material whose gradual erosion causes the gradual release of the αMSH to the brain tissue environment. In some embodiments, the sustained release device comprises a bioresorbable polymer. Preferably, the bioresorbable polymer has a half-life of at least six months, more preferably at least nine months, more preferably at least 12 months.

In some embodiments, the sustained release device provides controlled release. In others, it provides continuous release. In others, it provides intermittent release. In others, the sustained release device comprises a biosensor.

A. K. Burkoth, Biomaterials (2000) 21:2395-2404, the entire teaching of which are incorporated herein by reference, discloses a number of photopolymerizable anhydrides suitable as sustained release devices. The repeating unit of these anhydrides comprises a pair of diacid molecules linked by anhydride bonds that are susceptible to hydrolysis. Because the diacid molecules are hydrophobic, there is a limited diffusion of water into the polymer, and so the polymer is subject only to surface degradation (not bulk degradation). This is advantageous because the lifetime of the polymer will essentially correspond to the mass of the polymer.

In some embodiments, the photopolymerized anhydride is selected from the group consisting of polymers of methacrylated sebacic acid (MSA), methacrylated 1,6-bis(p-carboxyphenoxy) hexane (MCPH), 1,3-bis(p-carboxyphenoxy)propane (CPP), methacrylated cholesterol (MC), methacrylated stearic acid (MstA) and blends and copolymers therefrom.

In one embodiment, a copolymer of poly[bis(p-carboxyphenoxy)propane]anhydride and sebacic acid in a 50:50 to 20:80 formulation is used as the sustained release device.

In some embodiments, the photopolymerization is carried out by adapting a light source to the distal end of the delivery cannula that enters the cranium. In other embodiments, a photo-optic cable is used to transmit light energy into the precursor components that have been deposited in the brain. In other embodiments, light is transmitted through the skin (i.e, transcutaneously). In some embodiments thereof, a photobleaching initiating system is used.

In some embodiments, a linear polyanhydride is first dissolved in a monomer, and then photopolymerized to form a S-IPN of a photopolymerized anhydride. These are particularly desirable where increased resistance to hydroysis is desired. Accordingly, in some embodiments, the composition of the present invention comprises a S-IPN comprising a photopolymerized anhydride.

In some embodiments, poly(1,6-bis(p-carboxyphenoxy)hexane (PCPH) is used. This polymer has a degradation of about 496 days, and so is desirably used as the composition of the present invention.

Polymerization is preferably initiated using photoinitiators. Photoinitiators that generate an active species on exposure to UV light are well known to those of skill in the art. Active species can also be formed in a relatively mild manner from photon absorption of certain dyes and chemical compounds.

These groups can be polymerized using photoinitiators that generate active species upon exposure to UV light, or, preferably, using long-wavelength ultraviolet light (LWUV) or visible light. LWUV and visible light are preferred because they cause less damage to tissue and other biological materials than UV light. Useful photoinitiators are those, which can be used to initiate polymerization of the macromers without cytotoxicity and within a short time frame, minutes at most and most preferably seconds.

Exposure of dyes and co-catalysts such as amines to visible or LWUV light can generate active species. Light absorption by the dye causes the dye to assume a triplet state, and the triplet state subsequently reacts with the amine to form an active species, which initiates polymerization. Polymerization can be initiated by irradiation with light at a wavelength of between about 200-700 nm, most preferably in the long wavelength ultraviolet range or visible range, 320 nm or higher, and most preferably between about 365 and 514 nm.

Numerous dyes can be used for photopolymerization. Suitable dyes are well known to those of skill in the art. Preferred dyes include erythrosin, phloxime, rose bengal, thonine, camphorquinone, ethyl eosin, eosin, methylene blue, riboflavin, 2,2-dimethyl-2-phenylacetophenone, 2-methoxy-2-phenylacetophenone, 2,2-dimethoxy-2-phenyl acetophenone, other acetophenone derivatives, and camphorquinone. Suitable cocatalysts include amines such as N-methyl diethanolamine, N,N-dimethyl benzylamine, triethanol amine, triethylamine, dibenzyl amine, N-benzylethanolamine, N-isopropyl benzylamine. Triethanolamine is a preferred cocatalyst.

Photopolymerization of these polymer solutions is based on the discovery that combinations of polymers and photoinitiators (in a concentration not toxic to the cells, less than 0.1% by weight, more preferably between 0.05 and 0.01% by weight percent initiator) will crosslink upon exposure to light equivalent to between one and three mWatts/cm² applied to the skin of nude mice.

In some embodiments, the sustained delivery device comprises bioerodable macrospheres. The IL-10 is preferably contained in a gelatin (or water or other solvent) within the capsule, and is released to the brain tissue environment when the outer shell has been eroded. The device can include a plurality of capsules having outer shells of varying thickness, so that the sequential breakdown of the outer shells provides periodic release of the IL-10.

In some embodiments, the sustained delivery device comprises an inflammatory-responsive delivery system, preferably comprising bioerodable microspheres that are eroded by invading macrophages. This technology provides a high correspondence between physiologic inflammation of brain tissue environment and the release of the IL-10 into that environment. Preferably, the technology disclosed in Brown et al., Arthritis. Rheum. 1998 Dec. 41(12) pp., 2185-95 is selected.

In some embodiments thereof the αMSH is delivered intrathecally through a drug pump. In some embodiments, the sustained delivery device comprises the devices disclosed in U.S. Pat. No. 5,728,396 (“Peery”), the specification of which is incorporated by reference in its entirety.

In some embodiments, the sustained delivery device comprises a plurality (preferably at least one hundred) of water-containing chambers, each chamber containing αMSH. Each chamber is defined by bilayer lipid membranes comprising synthetic duplicates of naturally occurring lipids. The release of the αMSH can be controlled by varying at least one of the aqueous excipients, the lipid components, and the manufacturing parameters. Preferably, the formulation comprises no more than 10% lipid. In some embodiments, the Depofoam™ technology of Skyepharma PLC (located in London, United Kingdom) is selected.

In some embodiments, the sustained delivery device comprises a delivery system disclosed in U.S. Pat. No. 5,270,300 (“Hunziker”), the specification of which is incorporated by reference in its entirety.

In some embodiments, the sustained delivery device comprises the co-polymer poly-DL-lactide-co-glycolide (PLG). Preferably, the formulation is manufactured by combining the IL-10, the co-polymer and a solvent to form a droplet, and then evaporating the solvent to form a microsphere. The plurality of microspheres are then combined in a biocompatible diluent. Preferably, the αMSH is released from the co-polymer by its diffusion therethrough and by the biodegradation of the co-polymer. In some embodiments hereof, the ProLease™ technology of Alkermes (located in Cambridge, Mass.) is selected.

Hydrogels can also be used to deliver the αMSH in a time-release manner to the brain tissue environment. A “hydrogel” is a substance formed when an organic polymer (natural or synthetic) is set or solidified to create a three-dimensional open-lattice structure that entraps molecules of water or other solution to form a gel. The solidification can occur, e.g., by aggregation, coagulation, hydrophobic interactions, or cross-linking. The hydrogels employed in this invention rapidly solidify to keep the αMSH at the application site, thereby eliminating undesired migration from the brain tissue. The hydrogels are also biocompatible, e.g., not toxic, to cells suspended in the hydrogel.

A “hydrogel-αMSH composition” is a suspension of a hydrogel containing desired αMSH. The hydrogel-αMSH composition forms a uniform distribution of αMSH with a well-defined and precisely controllable density. Moreover, the hydrogel can support very large densities of αMSH. In addition, the hydrogel allows diffusion of nutrients and waste products to, and away from, the αMSH, which promotes tissue growth.

Hydrogels suitable for use in the present invention include water-containing gels, i.e., polymers characterized by hydrophilicity and insolubility in water. See, for instance, “Hydrogels”, pages 458-459 in Concise Encyclopedia of Polymer Science and Engineering, Eds. Mark et al., Wiley and Sons, 1990, the disclosure of which is incorporated herein by reference. Although their use is optional in the present invention, the inclusion of hydrogels is preferred since they tend to contribute a number of desirable qualities. By virtue of their hydrophilic, water-containing nature, hydrogels can house viable cells, such as mesenchymal stems cells.

In a preferred embodiment, the hydrogel is a fine, powdery synthetic hydrogel. Suitable hydrogels exhibit an optimal combination of such properties as compatibility with the matrix polymer of choice, and biocompatability. The hydrogel can include any of the following: polysaccharides, proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), and sulfonated polymers.

In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly(phosphazenes), poly(acrylic acids), and poly(methacrylic acids). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole).

In some embodiments, the sustained delivery device includes a polymer selected from the group consisting of PLA, PGA, PCL, and mixtures thereof.

In other embodiments, the αMSH is administered intrathecally.

In addition to stroke and Alzheimer's Disease, it is believed that the devices of the present invention would also be effective in treating other inflammatory mediated diseases of the central nervous system, including Infection (Viral, Bacterial, Protozoal, Prion, Fungal), AIDs-related Alzheimer's Disease, Multiple Sclerosis, Devic's disease and ADEM Sarcoidosis, acute brain injury, auto-immune diseases and vasculitides, and Paraneoplastic syndromes.

Modifications of the αMSH and its functional fragments that either enhance or do not greatly affect the ability to inhibit oxidation are also included within the term “αMSH”. “Such modifications include, for example, additions, deletions or replacements of one or more amino acids from the native amino acid sequence of αMSH with a structurally or chemically similar amino acid or amino acid analog. These modifications will either enhance or not significantly alter the structure, conformation or functional activity of the αMSH or a functional fragment thereof. Modifications that do not greatly affect the activity of the αMSH or its functional fragments can also include the addition or removal of sugar, phosphate or lipid groups as well as other chemical derivations known in the art. Additionally, αMSH or its functional fragments can be modified by the addition of epitope tags or other sequences that aid in its purification and which do not greatly affect its activity.

As used herein, the term “functional fragment,” in connection with αMSH, is intended to mean a portion of the αMSH that maintains the ability of the αMSH to inhibit inflammation. A functional fragment can be, for example, from about 6 to about 300 amino acids in length, for example, from about 7 to about 150 amino acids in length, more preferably from about 8 to about 50 amino acids in length. If desired, a functional fragment can include regions of the αMSH with activities that beneficially cooperate with the ability to inhibit inflammation.

I claim:

1. A method of providing therapy for a patient having a brain disorder, comprising the steps of:
a) selectively heating a first portion of the brain of the patient to induce cooling of a second portion of the brain.

2. The method of claim 1 wherein the heating induces intracerebral release of an effective amount of a antipyretic from the hypothalamus.

3. The method of claim 2 wherein the antipyretic is αMSH.

4. The method of claim 1 wherein the heated portion of the brain is the hypothalamus.

5. The method for claim 1 wherein the heated portion of the brain is the preoptic portion of the hypothalamus.

6. The method of claim 1 wherein the heated portion of the brain is the septum.

7. The method of claim 1 wherein the heating is accomplished by ultrasound.

8. The method of claim 1 wherein the heating is accomplished by microwave.

9. The method of claim 1 wherein the heating produces a local temperature rise in the heated potion of the brain of at least 1° C.

10. The method of claim 1 wherein the cooling is effective to produce a temperature drop of between about 1° C. and 10° C. in the second portion of the brain.

11. The method of claim 1 wherein the heating produces a local temperature in the heated portion of the brain of between 39° C. and 42° C.

12. The method of claim 1 comprising the further step of:
b) monitoring the temperature in the heated portion of the brain.

13. The method of claim 1 comprising the further step of:
b) monitoring the temperature in the cooled portion of the brain.

14. The method of claim 1 wherein the patient has suffered a stroke.

15. The method of claim 1 wherein the patient has a neurodegenerative disease.

16. The method of claim 1 wherein the patient has Alzheimer's Disease.

17. A method of providing therapy for a patient having a brain disorder, comprising the steps of:
a) selectively heating a portion of the brain to induce intracerebral release of MSH from the hypothalamus.

18. The method of claim 17 wherein the MSH is released into the septum in an amount effective to influence actylcholine turnover in the septo-hippocampal neurons.

19. The method of claim 17 wherein the patient has Alzheimer's Disease.

20. The method of claim 17 wherein the MSH is released into the hippocampus in an amount effective to attenuate inflammation therein.

21. The method of claim 17 wherein the MSH is released into the amygdala in an amount effective to attenuate inflammation therein.

22. The method of claim 17 wherein the patient has a neurodegenerative disease.

23. A device for providing therapy to the brain, comprising;
a) an ultrasound emitter adapted to heat a portion of the brain, and
b) a temperature monitor.

24. The device of claim 23 wherein the ultrasound emitter is adapted to heat the hypothalamus.

25. The device of claim 23 wherein the temperature monitor is adapted to monitor the temperature of the hypothalamus.
